(19) United States
(12) Patent Application Publication
(10) Pub. No.: US 2021/0177304 A1
(43) Pub. Date: Jun. 17, 2021

(54) SYSTEMS AND METHODS FOR DETECTING INFECTIONS

(71) Applicant: Avisa Pharma, Inc., Santa Fe, NM (US)

(72) Inventor: David L. KARSHMER, Santa Fe, NM (US)

(73) Assignee: Avisa Pharma, Inc., Santa Fe, NM (US)

(21) Appl. No.: 17/120,455

(22) Filed: Dec. 14, 2020

Related U.S. Application Data
(60) Provisional application No. 62/947,124, filed on Dec. 12, 2019.

(51) Int. Cl. A61B 5/097; A61B 5/08; G01N 33/497

(52) U.S. Cl. CPC A61B 5/097; G01N 33/497; A61B 5/082

(57) ABSTRACT

Provided herein are systems and methods for collecting breath samples for detection of respiratory infections.

### CROSS REFERENCE TO RELATED APPLICATIONS

This application claims priority under 35 U.S.C. § 119(e)(1) from U.S. Provisional Application Ser. No. 62/947,124, filed Dec. 12, 2019, the contents of which are incorporated herein by reference.

### FIELD

This disclosure is related to systems and methods for collecting breath samples for use in the detection of urease respiratory colonizations and infections.

### BACKGROUND

Early detection of whether a patient has a respiratory infection is important in providing suitable medical treatment for the patient and producing acceptable health outcomes. Respiratory infections not properly treated in a timely fashion can cause significant increases in length and cost of care as well as increased morbidity. Patients with community acquired and hospital associated pneumonia can suffer from both colonization and infection by virulent urease pathogens. Urease pathogens are actors in 5-15% of pneumonia patients entering hospital emergency rooms. Medical treatment for these pathogens commonly involves the use of broad spectrum antibiotics and hospital admission for observation of the resolution of the infection under antibiotic therapy. While this is appropriate treatment for 5-15% of these patients, the remaining 85-95% may unnecessarily receive exposure to broad spectrum antibiotics, a public health issue, and costly treatment in hospitals. Effectively identifying those patients who do not need broad spectrum antibiotics and hospitalization will relieve a burden on public health and patient welfare.

U.S. Pat. No. 9,518,972 describes methods of detecting bacterial infections by measuring ¹³CO₂/¹²CO₂ isotopic ratios of gaseous carbon dioxide in exhaled breath samples of a subject after administration of a ¹³C-isotopically-labeled compound that is metabolized by the urease pathogens.

### BRIEF DESCRIPTION OF THE DRAWINGS

FIG. 1A shows a system according to one embodiment described herein.

FIG. 1B shows another view of the system of FIG. 1A.

FIG. 1C shows another view of the system of FIG. 1A in which the sample collector is coupled to a nebulizer handset.

FIG. 2 shows the sample collector of the system of FIG. 1A connected to tubing.

FIG. 3 shows a top cross-sectional view of the sample collector of FIG. 2 and an inlet valve disposed in the inlet of the sample collector.

FIG. 4 shows a perspective view of the sample collector of FIG. 2.

FIG. 5 shows an end view of the sample collector of FIG. 2.

FIG. 6 shows a side view of the sample collector of FIG. 2 coupled to a nebulizer handset.

FIG. 7 shows a perspective view of the sample collector of FIG. 2 coupled to the nebulizer handset.

FIG. 8 shows a schematic view of a system according to one embodiment, including an analyzer, a sample collector and a mouthpiece assembly.

FIG. 9 shows a schematic view of a system according to another embodiment.

FIG. 10 is a flow diagram illustrating a method of collecting a breath sample, according to one embodiment.

### DETAILED DESCRIPTION

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

As used herein, use of a singular article such as “a,” “an” and “the” is not intended to exclude pluralities of the article's object unless the context clearly and unambiguously dictates otherwise. As used herein, the term “fluid” is used to describe the contents expelled by a subject during breathing and includes, predominantly, breath gases, but can also include liquids. Terms such as “fluidly connected” and “fluidly coupled” refer to a connection in which breath gases and/or liquids can be transferred between the connected or coupled components.

It can be difficult to detect respiratory infections prior to a subject becoming highly symptomatic. This can present problems in the timely treatment and care of such subjects. The systems and methods described herein overcome these difficulties and allow for the collection of breath samples from subjects for analysis of breath gases for a test marker (e.g., ¹³CO₂) indicating the presence of pathogens associated with infection and other health conditions. For example, the systems and methods described herein allow for the early detection of respiratory and other urease pathogen infections in pre-symptomatic and symptomatic patients and assessment of the presence and level of putative urease pathogens in the patient's respiratory system. Elevated levels of such pathogens can be associated with community acquired pneumonia (“CAP”), hospital acquired pneumonia (“HAP”), or ventilator associated pneumonia (“VAP”). Although the systems and methods described herein are well-suited to the detection of pneumonia, it should be understood that these systems and methods can additionally and/or alternatively be used to detect other infections, such as tuberculosis, cystic fibrosis, and others.

In various embodiments, the systems and methods described herein are configured for the collection of breath samples to allow for the detection of respiratory and systemic infections in a subject (e.g., a human patient) in conjunction with delivery of a drug to the subject. The drug may be configured to be metabolized by putative urease pathogens colonizing and/or infecting the subject. The metabolism of the drug by the putative urease pathogens produces elevations in the abundance of ¹³CO₂ in the patient's breath samples. In various embodiments, the described systems and methods involve collection of one or more baseline breath sample before introduction of the drug into the subject's respiratory airway, as well as one or more breath samples collected a selected period or periods after the completion of the drug delivery. Comparing the abundance of ¹³CO₂ in the post-administration sample(s) to the abundance of ¹³CO₂ in the baseline sample(s) allows for the detection of urea metabolizing infections of interest.

As described, breath samples are measured for changes in the abundance ratio of ¹³CO₂ reflective of the metabolism of ¹³C urea by the urease pathogens of specific clinical interest in pneumonia patients (CAP, HAP, VAP). Two breath samples are collected for measurement and comparison in the ¹³C urea breath test. The first breath sample is collected and measured to establish the baseline ¹³CO₂ abundance. A second sample is collected after delivery of ¹³C urea into the patient's respiratory tract. The change in breath ¹³CO₂ abundance between the baseline sample and the post exposure sample reflects the presence of urease pathogens present in either colonizations or infections. Accurate measurement of ¹³CO₂ abundance changes related to lower respiratory tract colonization or infection requires that breath samples represent ¹³CO₂ changes at or near the anatomical location of interest (e.g., lower respiratory tract), and not be confounded by signals that may arise in other areas of the respiratory tract. In particular, ¹³CO₂ signal produced by urease pathogens in the mouth and upper respiratory tract can be particularly problematic in making accurate measurements of changes in the lower respiratory tract. The breath collection systems described herein are configured to reduce or eliminate the amount of gases originating in the mouth or upper respiratory tract from the analyzed breath sample.

As described above, the methods described herein include administering to the subject a urea drug that includes an effective amount of a ¹³C-isotopically-labeled compound that produces ¹³CO₂ upon bacterial metabolism. Administration of the ¹³C-isotopically-labeled compound can be achieved by any appropriate means. For example, in one embodiment, the compound is administered via a nebulizer (e.g., a mesh nebulizer or a jet nebulizer). The ¹³C urea marker may also be delivered by dry powder inhaler, DPI, or metered dose inhaler (“MDI”).

Compositions for oral administration or inhalation of the ¹³C urea drug can be in any appropriate form. Oral compositions can include powders or granules, suspensions or solutions in water or non-aqueous media, sachets, capsules or tablets. Thickeners, diluents, flavorings, dispersing aids, emulsifiers or binders may be used. Compositions for pulmonary administration may include a pharmaceutically acceptable carrier, additive or excipient, as well as a propellant and optionally, a solvent and/or a dispersant to facilitate pulmonary delivery to the subject. Sterile compositions for injection can be prepared according to methods known in the art.

The ¹³C urea drug can be, for example, inhaled by the patient using a nebulizer affitted to a nebulizer handset, mouthpiece or mask. When the patient inhales from the nebulizer, breath is conducted into the patient's respiratory tract by normal breathing, and the drug is distributed to all parts of the respiratory tract. The presence of urease pathogens—that can be present in respiratory tract colonizations and infections—is not limited to the lower respiratory tract. These pathogens can also be found in the upper respiratory tract and mouth. The presence of such pathogens in the upper respiratory tract and mouth does not have the same clinical import as the presence of pathogens in the lower respiratory tract. For example, the patient can have active mouth colonization of urease pathogens that does not correlate to a lower respiratory tract infection. As a result, the metabolism of ¹³C urea by urease pathogens in the mouth can produce a confounding quantity of ¹³CO₂ that prevents a breath sample from providing a reliable indication of lower respiratory tract infections. As described in further detail herein, in order to more accurately understand the colonization and or infection of the lower respiratory tract, the lower respiratory tract sample may be separated, or fractionated, from the sample that originates in the mouth and upper respiratory tract. Fractionation of the exhaled breath to collect a more representative lower respiratory tract sample reduces potential confounding mouth and upper respiratory signals. Doing so produces ¹³C urea breath tests that more clearly represent the presence of urease pathogens in the lower respiratory tract.

Any bacteria that can convert the ¹³C-isotopically-labeled compound administered to the subject into ¹³CO₂ can be detected using the systems and methods described herein. Examples of such bacteria include Pseudomonas aeruginosa, Staphylococcus aureus, Mycobacterium tuberculosis, Acenitobacter baumannii, Klebsiella pneumonia, Francisella tularenis, Proteus mirabilis, Aspergillus species, and Clostridium difficile.

The detection apparatus for analyzing the breath samples can include near infrared diode lasers to attain field portable, battery operated δ¹³CO₂ measurement instruments with high degrees of accuracy and sensitivity. These devices and the methodologies which employ them may be used to determine δ¹³CO₂ in exhaled breath samples of subjects having, or suspected of having, a bacterial infection. The analyzer can include features and analyze the sample as described in U.S. Pat. No. 9,518,972, which is incorporated herein by reference in its entirety.

This disclosure provides devices and methods for collecting breath samples from subjects such that only a portion of the breath sample is retained and analyzed. Such devices and methods can be used, for example, to preferably retain portions of breath sample that originate in the lower respiratory tract and discard portions that originate in the upper respiratory tract and mouth. The devices and method for collecting breath samples may be used, for example, in subjects in which community acquired or hospital acquired pneumonia is suspected. As described further herein, the volume of breath sample retained can be selected to retain the desired portion of the subject's exhaled breath gases.

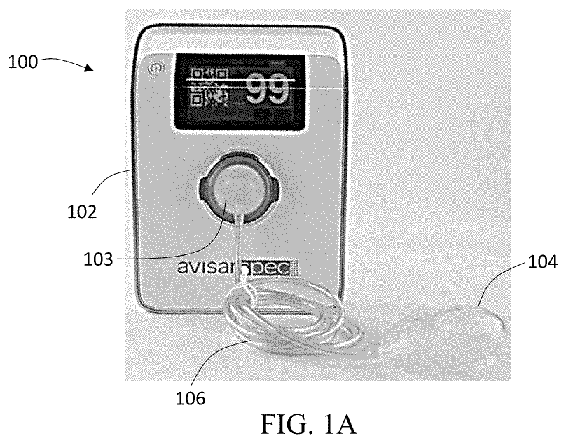

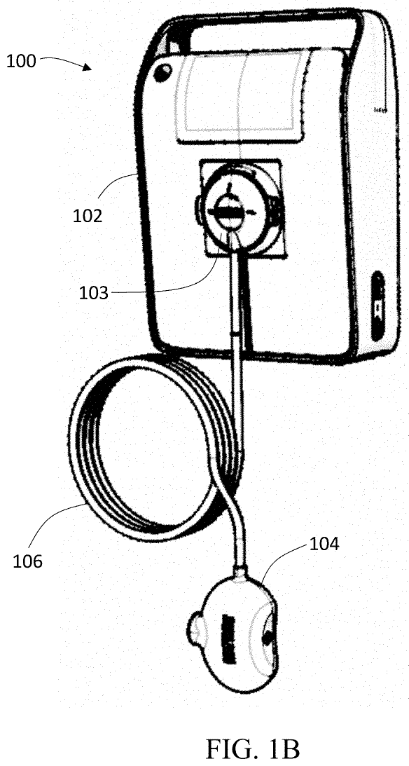

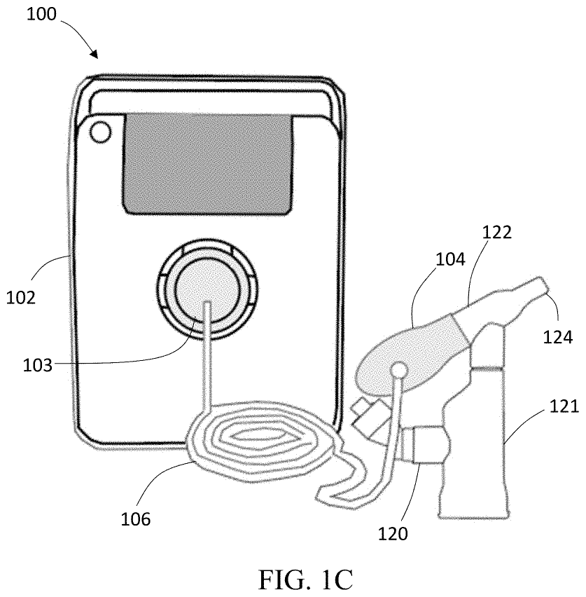

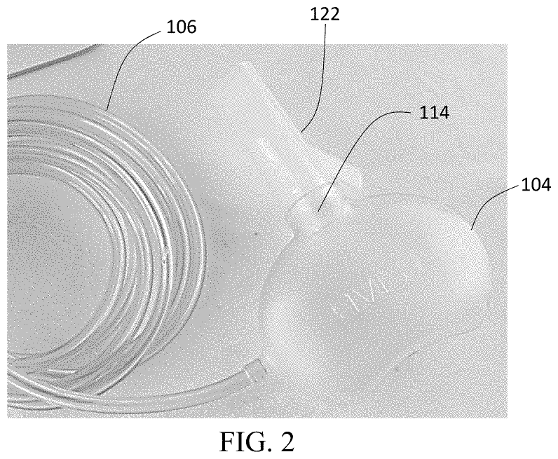

FIGS. 1A, 1B, 1C, and 2 show a system 100 for collecting and analyzing breath samples of a subject. The system 100 includes an analyzer 102 and a sample collector 104. As shown, the analyzer 102 and the sample collector 104 are fluidly connected by tubing 106. The analyzer 102 can include a spectrometer. For example, the analyzer 102 can be an AVISAR™ spectrometer distributed by Avisa Pharmaceuticals, Inc. The tubing 106 can be, for example, PVC tubing with a ⅛″ inner diameter. The analyzer 102 can include a biologic filter 103 to which the tubing 106 connects. The filter 103 can be configured, for example, to prevent biologic microparticulate from entering the analyzer 102. As shown in FIG. 1C, and described in more detail below, the sample collector 104 can be configured to couple to an exit port 122 of a mouthpiece 124 coupled to a nebulizer handset 121.

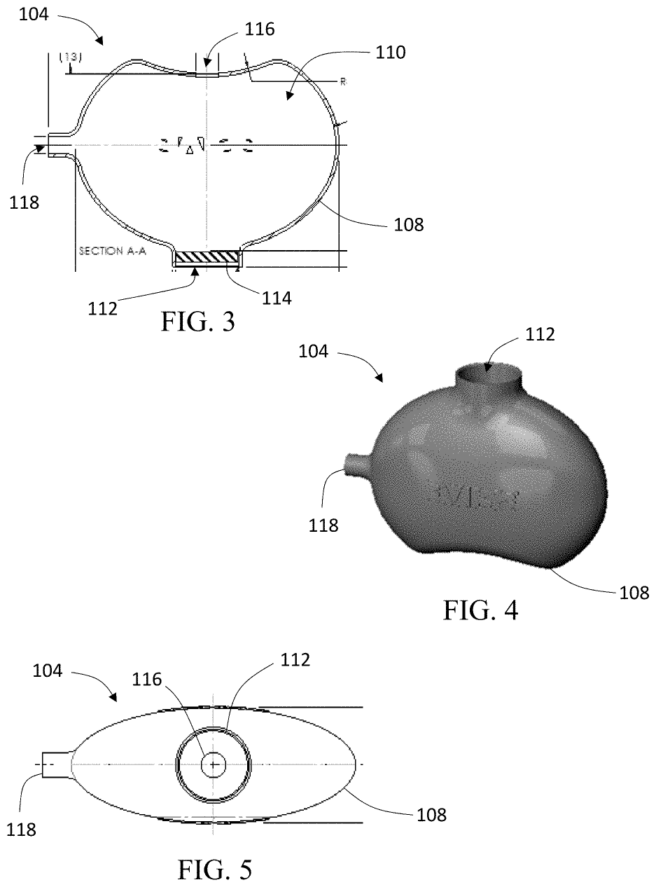

As shown in FIGS. 3-5, the sample collector 104 includes a body 108 defining a breath collection reservoir chamber 110. As will be described further herein, the reservoir chamber 110 is configured to receive a breath sample of the subject. The body 108 can be constructed of any appropriate material, such as, for example, polypropylene or other polymer. In other embodiments, the sample collector 104 is constructed from Tedlar. In some embodiments, the interior of portions of the body 108 can be coated with a hydrophilic coating to remove moisture from the breath sample in order to reduce the quantity of moisture that is introduced to the analyzer 102. Additionally, or alternatively, the tubing 106 can also include a hydrophilic coating to reduce the amount of moisture introduced into the analyzer 102.

The body 108 further defines an inlet 112 opening into the reservoir chamber 110. As shown in FIGS. 2 and 3, an inlet valve 114, such as a one-way valve, is disposed in the inlet 112. The inlet valve 114 controls the flow of air through the inlet 112 and into the reservoir chamber 110. Specifically, in use, the inlet valve 114 is configured to open at the beginning of the subject's exhalation and close at the end of the exhalation. Hence, the inlet valve 114 only allows fluid to flow into the reservoir chamber 110 during an exhalation of the subject. In this way, the inlet valve 114 prevents ambient air from entering into the reservoir chamber 110. The inlet valve 114 also prevents fluid from flowing out of the reservoir chamber 110 and through the inlet 112 during inhalation by the subject. While the inlet valve 114 is described herein as being positioned in the inlet 112 of the body 108, in other embodiments (not shown) the inlet valve 114 is disposed in a nebulizer or nebulizer handset that the sample collector 104 is coupled to (e.g., in the exit port 122). The inlet valve 114 can be any type of one way valve, such as, for example, an umbrella valve. The inlet valve 114 can preferably have a low cracking (i.e., opening) pressure to reduce back-pressure exerted on the patient's breath. The inlet valve 114 is configured to close after completion of exhalation and before initiation of the subject's next inhalation to prevent the flow of fluids out of the reservoir chamber 110 and into the subject's mouth.

The sample collector 104 is configured such that only sample from the desired portion of the subject's exhalation is retained in the reservoir chamber 110. For example, for purposes of identifying infections in the lower respiratory tract of the subject, the initial portion of the patient's exhalation may not be retained in the reservoir chamber 110. This portion of the exhalation may originate from the mouth and upper respiratory tract and, therefore, may not be indicative of infections in the lower respiratory tract.

In order to discard the fluid from the early portion of the subject's exhalation, the sample collector 104 can include a purge aperture 116. As shown in FIG. 3, the purge aperture 116 may be on the opposite portion of the body 108 from the inlet 112. In some embodiments, the purge aperture 116 is open to the environment. In other embodiments, flow through the purge aperture 116 is restricted by a flow restrictive structure such as a filter or valve. During use, when the patient exhales, fluid flows through the inlet 112 and into the reservoir chamber 110. The purge aperture 116 may be configured such that it introduces a low flow resistivity to allow convective flow through the purge aperture 116. The size of the purge aperture 116 may be chosen to balance the goals of reducing passive diffusion of the sample through the purge aperture 116 while also minimizing back pressure on the patient's breathing. CO₂ has a very low diffusion constant in open air, thereby helping to retain the sample in the reservoir chamber 110 during the patient's inhalation. The cross-sectional area of the purge aperture 116 is preferably smaller than the cross-sectional area of the inlet 112 to restrict the flow of fluids out through the purge aperture 116 during breath sample collection. In some embodiments, the purge aperture 116 is circular and has a diameter of about 9 mm. In another embodiment, the purge aperture 116 has a diameter of about 7 mm and about 11 mm.

The volume of the reservoir chamber 110 is configured to be less than the total exhaled volume of the patient. As a result, the fluid that enters the reservoir chamber 110 at the beginning of exhalation is forced out through the purge aperture 116 as exhalation continues and more fluid flows into and through the reservoir chamber 110. For example, in some embodiments, the volume of the reservoir chamber 110 is about 150 ml (milliliters). In another embodiment, the volume of the reservoir chamber 110 is between about 125 ml and about 175 ml. In another embodiment, the volume of the reservoir chamber 110 is between about 100 ml and about 200 ml. In another embodiment, the volume of the reservoir chamber 110 is about 300 ml. In another embodiment, the volume of the reservoir chamber 110 is between about 50 ml and 300 ml.

The tidal volume for a patient is typically between about 350 ml and about 700 ml. Because the volume of the reservoir chamber 110 is less than the tidal volume, the fluid from the first portion of expiration is forced out of the reservoir chamber 110 by fluid that subsequently enters the reservoir chamber 110. Further, as the subject takes additional breaths, the fluid exhaled during the subsequent breaths displaces the fluid that is present within the reservoir chamber 110. In these subsequent breaths, the fluid that is exhaled at the later portions of the exhalation displaces the fluid from the initial portion of the exhalation, as described above.

The body 108 further defines an outlet 118. The tubing 106 is connected to the outlet 118 to allow the flow of fluid from the reservoir chamber 110 to the analyzer 102. In some embodiments, the analyzer 102 includes a spectrometry chamber that is at a pressure that is, in use, less than the pressure within the reservoir chamber 110. For example, the spectrometry chamber may be at a pressure of about 75 to 375 Torr. As a result, the fluid flows from the reservoir chamber 110 to the spectrometry chamber through the tubing 106. In other embodiments, the sample collector 104 is directly coupled to the analyzer 102.

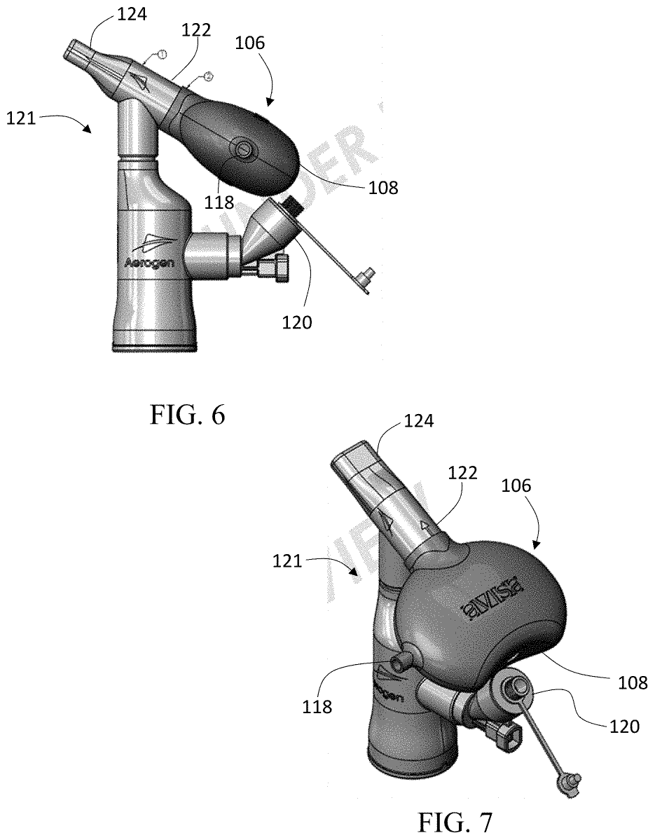

In some embodiments, as shown in FIGS. 6-7, the sample collector 104 is configured to couple to a nebulizer handset 121 (e.g., to the exit port 122 of the mouthpiece 124 of the nebulizer handset 121). A nebulizer 120 is also coupled to the nebulizer handset 121 to allow for the delivery of a drug to the subject. The subject may breathe using the nebulizer handset 121 such that the drug is delivered from the nebulizer 120 during an inhalation phase of the subject's respiratory cycle. As the subject exhales, the fluid expelled from the subject's respiratory tract flows through the mouthpiece 124 of the nebulizer handset 121, through the exit port 122, and into the sample collector 104 where fluid from the desired portion of the exhalation is retained, as described above. In some embodiments, the nebulizer handset 121 is an AEROGEN ULTRA sold by Aerogen of Galway, Ireland and the nebulizer 120 is an AEROGEN SOLO sold by the same company. The geometry of the sample collector 104 can be configured to accommodate the nebulizer handset 121 and the nebulizer 120. For example, as shown in FIG. 7, the portion of the body 108 near the purge aperture 116 can be concave to allow access to the nebulizer 120.

It should be understood that the sample collector 104 need not be connected to the nebulizer handset 121. In some embodiments, the sample collector 104 is connected to a dedicated mouthpiece or mask with appropriate valve(s) to control the flow into the reservoir chamber 110, as described herein.

The rate of transport of breath gases from the reservoir chamber 110 to the analyzer 102 can be controlled to ensure that the pressure within the reservoir chamber 110 is maintained within a desired range that prevents the flow of ambient air into the reservoir chamber, which would result in dilution of the sample. In other words, the pressure in the reservoir chamber is maintained at or above ambient air pressure. The rate of emptying of the reservoir chamber 110, and thereby the pressure in the reservoir chamber 110, can be controlled by controlling the flow into the analyzer 102 (e.g., by controlling the pressure in the spectrometry chamber or using a variable restriction valve) as well as through appropriate selection of the length and diameter of the tubing 106. This may prevent cracking of the body 108 and opening of the inlet valve 114. In some embodiments, fluid is drawn continuously over a nine second period. This may equate to breath sample entering the analyzer 102 at a rate of 33.3 ml/second. This rate can be modified to optimize system performance if different reservoir volumes are selected, or if total sample volume required by the spectrometer is changed. During the sample collection period, the patient may exhale 3-5 breaths. As a result, the sample that is analyzed represents a blend of the gas exhaled during these breaths. With each breath, the portion of breath sample analyzed preferably originates from the lower respiratory tract as a result of the arrangement of the purge aperture 116, as described above.

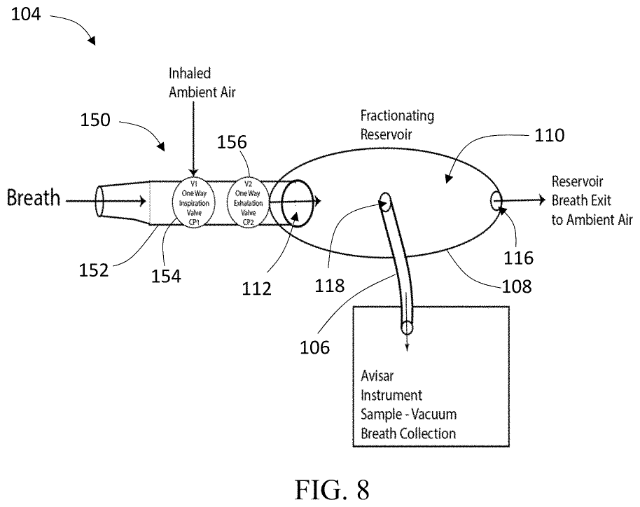

In some embodiments, as shown in FIG. 8, the inlet 112 of the sample collector 104 is connected to a mouthpiece assembly 150 as opposed to a nebulizer handset 121. In this dedicated breath collection assembly, the mouthpiece assembly 150 includes a mouthpiece 152, an inspiration valve 154, and an exhalation valve 156. Each of the valves 154, 156 are one-way valves. The inspiration valve 154 can be configured to allow the flow of ambient air into the mouthpiece during inspiration. The exhalation valve 156 is configured to allow for the passage of exhalation fluids through the mouthpiece 152 and into the reservoir chamber 110.

In such embodiments, when the subject breathes in, the pressure within the mouthpiece 152 may decrease, thereby causing the inspiration valve 154 to open to allow ambient air to flow into the mouthpiece 152. When the subject breathes out, the pressure within the mouthpiece 152 increases, thereby closing the inspiration valve 154 and opening the exhalation valve 156 to allow fluid to flow into the reservoir chamber 110. As described above, the volume of the reservoir chamber 110 in conjunction with the purge aperture 116 leads to only the desired portion of the exhalation to be retained in the reservoir chamber 110.

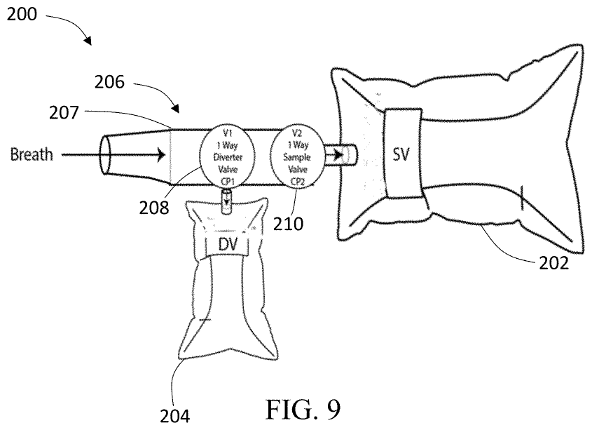

In another embodiment, shown in FIG. 9, a sample collector 200 includes a first body 202 to collect the sample of interest, a second body 204 to collect fluid from the first part of exhalation (i.e., from the mouth and upper respiratory tract), and a valve apparatus 206. The valve apparatus 206 includes a tube 207, a first diverter valve 208 and a second sample valve 210. Each of the valves 208, 210 are disposed within, or coupled to, the tube 207. The tube 207 defines a lumen for the passage of the fluid from the breath of a subject. Each of the first body 202 and the second body 204 is in fluid communication with the lumen of the tube 207. During the first part of exhalation, the valves 208, 210 are configured such that the fluid from the exhalation flows from the lumen of the tube 207 into the second body 204. At the desired time, the valves 208, 210 are reconfigured such that fluid from the latter part of the exhalation flows from the lumen of the tube 207 into the first body 202. As a result, only the desired portion of the exhalation fluid is retained in the first body 202. This can be ensured by selecting the valves 208, 210 such that the first valve 208 has a lower cracking (i.e., opening) pressure than the second valve 210. Because the first valve 208 has a lower cracking pressure than the second valve 210, breath will enter the second body 204 until the second body 204 is filled. At that point, the first valve 208 will close, thereby allowing air to pass through the tube 207, through the second valve 210, and into the first body 202. The sample from the first body 202 can be transported to the analyzer 102 for analysis. For example, the sample can travel through tubing coupled to the first body 202 as samples are collected, similar to the tubing 106 described above with reference to FIGS. 1A-1C. Alternatively, the breath sample can first be collected in the first body 202 and subsequently introduced to the analyzer 102 by connecting the first body 202 to the analyzer 102 (either directly or through tubing) after collection of the breath samples is complete.

As noted above, the selective collection of breath gases in the first body 202 may be accomplished through appropriate selection of the cracking pressure of the valves 208, 210. Alternatively, the opening and closing of the valves 208, 210 can be operated manually to collect the desired portion of exhalation gases. Alternatively, or additionally, the valve apparatus 206 can include sensors to sense patient breathing or pressure or flow changes within the valve apparatus 206 and/or the first body 202 or second body 204. In this way, the valves 208, 210 can be automatically operated to collect the desired portion of the exhalation. For example, the sensor can communicate with a microcontroller that controls the position or configuration of the valves 208, 210 (i.e., whether the valves are opened or closed).

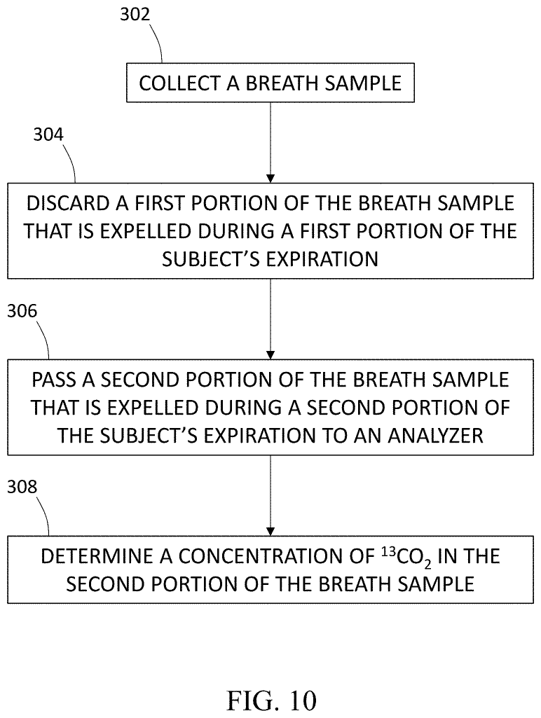

In another aspect, FIG. 10 illustrates a method of collecting and analyzing a breath sample. At step 302, a breath sample is collected. At step 304, a first portion of the breath sample is discarded. The first portion of the breath sample is expelled during a first portion of the subject's expiration. At step 306, a second portion of the breath sample is passed to an analyzer. The second portion of the breath sample is expelled during the second portion of the subject's expiration and the second portion occurs subsequent to the first portion.

The first portion of the breath sample preferably includes fluid that originates from the patient's mouth and upper respiratory tract and the second portion of the breath sample preferably includes fluid that originates in the lower respiratory tract. For example, the first portion of the breath sample (i.e., the portion that is discarded) can include breath gases from the first 17%-93% of the duration of the patient's tidal volume.

The methods described herein can include capturing and analyzing breath samples before and after administration of a drug (e.g., a ¹³C urea drug). The samples collected prior to administration of the drug serve as a baseline to which the post-administration samples can be compared. Both the pre-administration and post-administration samples can be fractionated, as described herein, such that the samples preferably include breath gases that originate from the lower respiratory tract. As described above, the breath samples, both before and after drug administration, can include fluid from one or more than one exhalations by the subject. The method can also include comparing the concentration of ¹³CO₂ in the breath samples collected before and after administration of the drug.

It will be understood that the foregoing description is of exemplary embodiments of this invention, and that the invention is not limited to the specific forms shown. Modifications may be made in the design and arrangement of the elements without departing from the scope of the invention.

What is claimed is:

1. A system for collection and analysis of breath samples, the system comprising:
an analyzer;
a sample collector, comprising:
a body defining a reservoir chamber, an inlet, a purge aperture, and an outlet, wherein each of the inlet, the purge aperture, and the outlet is in fluid communication with the reservoir chamber, and wherein the outlet is configured to be fluidly coupled to the analyzer; and
an inlet valve configured to control flow of fluid through the inlet;
wherein the reservoir chamber is configured to receive a breath sample introduced through the inlet such that a first portion of the breath sample exits the reservoir chamber through the purge aperture and a second portion of the breath sample exits the reservoir chamber through the outlet to flow to the analyzer.

2. The system of claim 1, wherein the reservoir chamber has a volume between 50 milliliters and 300 milliliters.

3. The system of claim 1, wherein the inlet of the body is configured to couple to a nebulizer handset.

4. The system of claim 1, wherein the purge aperture is circular and the diameter of the purge aperture is about 9 mm.

5. The system of claim 1, wherein the purge aperture has a cross-sectional area that is less than a cross-sectional area of the inlet.

6. The system of claim 1, further comprising a mouthpiece assembly coupled to the inlet of the body of the sample collector, the mouthpiece assembly comprising:
a mouthpiece having a mouthpiece opening;
an inspiration valve configured to control flow of ambient air into the mouthpiece and through the mouthpiece opening; and
an exhalation valve configured to control flow of fluid exhaled by a patient into the mouthpiece opening and into the reservoir chamber.

7. A sample collector, comprising:
a body defining a reservoir chamber, an inlet, a purge aperture, and an outlet, wherein each of the inlet, the purge aperture, and the outlet is in fluid communication with the reservoir chamber; and
an inlet valve configured to control flow of fluid through the inlet;
wherein the reservoir chamber is configured to receive a breath sample introduced through the inlet such that a first portion of the breath sample exits the reservoir chamber through the purge aperture and a second portion of the breath sample exits the reservoir chamber through the outlet.

8. The sample collector of claim 7, wherein the reservoir chamber has a volume between 50 milliliters and 300 milliliters.

9. The sample collector of claim 7, wherein the inlet of the body is configured to couple to a nebulizer handset.

10. The sample collector of claim 7, wherein the purge aperture is circular and the diameter of the purge aperture is about 9 mm.

11. The sample collector of claim 1, wherein the purge aperture has a cross-sectional area that is less than a cross-sectional area of the inlet.

12. The sample collector of claim 7, further comprising a mouthpiece assembly coupled to the inlet of the body, the mouthpiece assembly comprising:
a mouthpiece having a mouthpiece opening;
an inspiration valve configured to control flow of ambient air into the mouthpiece and through the mouthpiece opening; and
an exhalation valve configured to control flow of fluid exhaled by a patient into the mouthpiece opening and into the reservoir chamber.

13. A sample collector, comprising:
a first body defining a sample chamber;
a second body defining a diverter chamber; and
a valve apparatus, comprising:
a tube defining a lumen for passage of breath of a subject, wherein the sample chamber and the diverter chamber are in fluid communication with the lumen of the tube; and
a first valve configured such that in a first configuration the first valve allows flow of fluid from the lumen to the diverter chamber and in a second configuration the first valve prevents the flow of fluid from the lumen to the diverter chamber.

14. The sample collector of claim 13, wherein the valve apparatus further comprises a second valve configured to allow fluid to flow from the lumen to the sample chamber and prevent fluid from flowing from the sample chamber to the lumen.

15. A method, comprising:
collecting a breath sample from a subject;
discarding a first portion of the breath sample that is expelled during a first portion of the subject's expiration; and
passing a second portion of the breath sample that is expelled during a second portion of the subject's expiration to an analyzer, wherein the second portion occurs subsequent to the first portion.

16. The method of claim 15, further comprising determining a concentration of ¹³CO₂ in the second portion of the breath sample.

17. The method of claim 15, wherein discarding the first portion comprises passing the first portion of the breath sample through a purge aperture of a body.

18. The method of claim 17, wherein the body defines a chamber for retaining the breath sample, and wherein the chamber has a volume of between 50 milliliters and 300 milliliters.
